SEQUENCE LISTING (1 sequence)

SEQ ID NO: 1 - protein, 9 aa, Homo sapiens
SFEDIHHYR

(19) United States
(12) Patent Application Publication
(10) Pub. No.: US 2019/0293652 A1
(43) Pub. Date: Sep. 26, 2019

(54) Quantifying KRAS for Optimal Cancer Therapy

(71) Applicant: EXPRESSION PATHOLOGY, INC., Rockville, MD (US)

(72) Inventors: Fabiola CECCHI, Potomac, MD (US); Adele BLACKLER, Denver, CO (US); Wei-Li LIAO, Herndon, VA (US); Todd HEMBROUGH, Gaithersburg, MD (US); Daniel CATENACCI, Chicago, IL (US)

(73) Assignees: Expression Pathology, Inc., Rockville, MD (US); NantOmics, LLC, Culver City, CA (US)

(21) Appl. No.: 16/071,815

(22) PCT Filed: Jan. 20, 2017
(86) PCT No.: PCT/US2017/014371

§ 371 Date: Jul. 20, 2018

Related U.S. Application Data
(60) Provisional application No. 62/281,266, filed on Jan. 21, 2016.

(51) Int. Cl. G01N 33/574; C07K 16/28

(52) U.S. Cl. CPC G01N 33/57407; G01N 2333/914; C07K 16/2863

(57) ABSTRACT

Methods are provided for identifying whether a tumor will be responsive to treatment with an anti-EGFR agent. Specific protein fragment peptides are precisely detected and quantitated by SRM-mass spectrometry directly in tumor cells collected from tumor tissue that was obtained from a cancer patient and compared to reference levels in order to determine if the lung cancer patient will positively respond to treatment with an anti-EGFR agent such as, for example, pamitumumab and/or erbitux.

### CROSS-REFERENCE TO RELATED APPLICATIONS

This application is a National Stage Entry of PCT/US2017/14371, filed Jan. 20, 2017, which claims the benefit of U.S. Provisional Application No. 62/281,266, filed Jan. 21, 2016, the contents of which are hereby incorporated by reference in their entirety.

### REFERENCE TO A SEQUENCE LISTING

This application contains references to amino acid sequences and/or nucleic acid sequences which have been submitted concurrently herewith as the sequence listing text file 62346671_1.TXT file size 1 KB, created on 25 Mar. 2019. The aforementioned sequence listing is hereby incorporated by reference in its entirety pursuant to 37 C.F.R. § 1.52(e)(5).

### INTRODUCTION

New and improved methods for treating cancer patients are provided. The methods provide assays for measuring the level of a specific protein in tumor tissue from patients, and then, based upon those levels, treat the patients with an optimized medication regimen. More specifically, a quantitative assay of KRAS in tumor tissue is provided and used to identify those patients that will respond to, or are most likely to respond to, treatment with regimens that include EGFR-targeted therapies, and anti-EGFR monoclonal antibody therapies in particular. The method is particularly useful for treating patients suffering from gastroesophageal adenocarcinoma.

The quantitative levels of KRAS protein expression in the tumor tissue are determined by quantitating a specified fragment peptide derived from the full-length proteins in digests prepared from formalin-fixed tissue samples. The quantitative level of KRAS can be measured using specific fragment peptides derived from KRAS as described in PCT/US2015/040208 (filed Jul. 13, 2015), the contents of which are hereby incorporated by reference in their entirety. If expression of the KRAS protein above a specified reference level is detected, the patient is treated with a regimen that does not include one or more anti-EGFR agents. Alternatively, if the KRAS level is below the reference level then the patient is treated with a regimen that includes at least one anti-EGFR agent, such as cetuximab, panitumumab, zalutumumab, nimotuzumab, and/or matuzumab.

The specified KRAS fragment peptide is detected using mass spectrometry-based Selected Reaction Monitoring (SRM), also referred to as Multiple Reaction Monitoring (MRM), and referred to herein as an SRM/MRM assay. An SRM/MRM assay is used to detect the presence and quantitatively measure the amount of the specified fragment peptides directly in cells procured from cancer patient tissue, such as, for example formalin fixed cancer tissue. The amount of the specific KRAS peptide is then used to quantitate the amount of full-length protein in the tumor sample. Specific and optimized therapeutic agents and treatment strategies are then determined and implemented to treat an individual cancer patient's disease based on how much of the KRAS protein is present in their cancer cells.

### SUMMARY OF THE INVENTION

Methods are provided for treating a patient suffering from cancer, such as gastroesophageal adenocarcinoma, comprising:

(a) quantifying the level of a specified KRAS fragment peptide in a protein digest prepared from a tumor sample obtained from the patient and calculating the level of the KRAS peptide in the sample by selected reaction monitoring using mass spectrometry;

(b) comparing the level of the KRAS fragment peptide to a reference level, and

(c) treating the patient with a therapeutic regimen that includes an effective amount of at least one anti-EGFR agent when the level of the KRAS fragment peptide is lower than the reference level, and

(d) treating the patient with a therapeutic regimen that does not include an effective amount of at least one anti-EGFR agent when the level of the KRAS fragment peptide is above the reference level.

In these methods the reference level of the KRAS fragment peptide may be, for example, 1331 amol/μg, +/−250 amol/μg, +/−150 amol/μg, +/−100 amol/μg, +/−50 amol/μg, or +/−25 amol/μg, of biological sample protein analyzed.

The protein digest may be a protease digest, such as a trypsin digest, prepared, for example, using the Liquid Tissue® protocol.

The mass spectrometry may be, for example, tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry. The mode of mass spectrometry used may be, for example, Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

In these methods the specified KRAS peptide may have the amino acid sequence as set forth as SEQ ID NO:1. The tumor sample may be, for example, a cell, collection of cells, or a solid tissue. The tumor sample may be formalin fixed solid tissue, and may be paraffin embedded tissue.

In these methods quantifying the specified KRAS fragment peptide may include determining the amount of the KRAS peptide in the sample by comparing to a spiked internal standard peptide of known amount, where both the native peptide in the biological sample and the internal standard peptide corresponds to the same amino acid sequence of the KRAS fragment peptide as shown in SEQ ID NO:1. The internal standard peptide may be, for example, an isotopically labeled peptide. The isotopically labeled internal standard peptide may comprise one or more heavy stable isotopes selected from ¹⁸O, ¹⁷O, ¹⁵N, ¹³C, ²H or combinations thereof.

Detecting and quantitating the specified KRAS fragment peptide can be combined with detecting and quantitating other peptides from other proteins in a multiplex format so that the treatment decision about which agent used for treatment is based upon specific levels of the specified KRAS fragment peptide, in combination with other peptides/proteins in the biological sample. When the level of the specified KRAS peptide is lower than the reference level, then the therapeutic strategy may include one or more anti-EGFR agents.

In the methods for detecting KRAS, the method may further include the step of fractionating the protein digest prior to detecting and/or quantifying the amount of the fragment peptide. Quantifying the fragment peptide may include comparing the amount of the fragment peptide in one biological sample to the amount of the same fragment peptide in a different and separate biological sample, or may include determining the amount of the fragment peptide in a biological sample by comparison to an added internal standard peptide of known amount, where the fragment peptide in the biological sample is compared to an internal standard peptide having the same amino acid sequence; and where the internal standard peptide is an isotopically labeled peptide.

In these methods detecting and/or quantifying the amount of the fragment peptide in the protein digest indicates the presence of the corresponding modified or unmodified protein and an association with cancer in the subject, and the results may be correlated to the diagnostic stage/grade/status of the cancer. The correlating step may be combined with detecting and/or quantifying the amount of other proteins or peptides from other proteins in a multiplex format to provide additional information about the diagnostic stage/grade/status of the cancer.

After the measurement and, optionally, the correlating step, the patient from which the biological sample was obtained is administered a therapeutically effective amount of a therapeutic agent, where the therapeutic agent and/or amount of the therapeutic agent administered is based upon the amount of the fragment peptide or the level of the protein.

When the level of the specified KRAS peptide is lower than the reference level then the therapeutic strategy may include at least one agent selected from the group consisting of the cetuximab, panitumumab, zalutumumab, nimotuzumab, and matuzumab.

In the methods described above, the patient may be suffering from gastroesophageal adenocarcinoma.

### DETAILED DESCRIPTION

Methods are provided for identifying and optimizing treatment strategies for a cancer patient by determining whether or not a cancer patient will clinically respond in a favorable manner to one or more anti-EGFR therapeutic cancer agents such as panitumumab, cetuximab, zalutumumab, nimotuzumab, and/or matuzumab. Specifically, diagnostic methods for measuring expression of KRAS in a tumor sample or samples from the patient are provided. The tumor sample is advantageously a formalin-fixed sample. Using an SRM/MRM assay that measures a specific KRAS peptide fragment, and particular characteristics about the peptide, the amount of the KRAS protein in cells derived from formalin fixed paraffin embedded (FFPE) tissue is determined. The peptide fragments derive from the full-length proteins; the specific peptide sequence that is used for KRAS is SFEDIHHYR (SEQ ID NO:1). Detection and accurate quantitation of this peptide in digests of FFPE tissue is highly unpredictable, due to the random protein crosslinking that occurs during formalin fixation of proteins. Surprisingly, however, it has been found that this specific KRAS peptide can be reliably detected and quantitated in digests prepared from FFPE samples of tumor tissue. See, for example, U.S. patent application Ser. No. 13/993,045, the contents of which are hereby incorporated by reference in their entirety.

More specifically, this SRM/MRM assay can measure the peptides directly in complex protein lysate samples prepared from cells procured from patient tissue samples, such as formalin fixed cancer patient tissue. Methods of preparing protein samples from formalin-fixed tissue are described in U.S. Pat. No. 7,473,532, the contents of which are hereby incorporated by reference in their entirety. The methods described in U.S. Pat. No. 7,473,532 may conveniently be carried out using Liquid Tissue® reagents and protocol available from Expression Pathology Inc. (Rockville, Md.).

The most widely and advantageously available form of tissue, and cancer tissue, from cancer patients is formalin fixed, paraffin embedded tissue. Formaldehyde/formalin fixation of surgically removed tissue is by far the most common method of preserving cancer tissue samples worldwide and is the accepted convention in standard pathology practice. Aqueous solutions of formaldehyde are referred to as formalin. “100%” formalin consists of a saturated solution of formaldehyde (about 40% by volume or 37% by mass) in water. A small amount of stabilizer, usually methanol, is added to limit oxidation and degree of polymerization. The most common way in which tissue is preserved is to soak whole tissue for extended periods of time (8 hours to 48 hours) in aqueous formaldehyde, commonly termed 10% neutral buffered formalin, followed by embedding the fixed whole tissue in paraffin wax for long term storage at room temperature.

Results from the SRM/MRM assay can be used to correlate accurate and precise quantitative levels of the KRAS protein within the specific cancer of the patient from whom the tissue was collected and preserved, including lung cancer tissue. This not only provides diagnostic information about the cancer, but also permits a physician or other medical professional to determine appropriate therapy for the patient. In this case, utilizing this assay can provide information about specific levels of KRAS protein expression in cancer tissue from a patient and makes it possible to determine whether or not the patient will respond favorably to therapy with the anti-EGFR agents that specifically inhibit the function of EGFR.

The KRAS protein is a GTPase that performs an essential function in normal tissue signaling, and mutation of the KRAS gene is an essential step in the development of many cancers.

The most widely-used methodology presently applied to determine protein presence in cancer patient tissue, especially FFPE tissue, is immunohistochemistry (IHC). IHC methodology uses an antibody to detect the protein of interest. The results of an IHC test are most often interpreted by a pathologist or histotechnologist. This interpretation is subjective and does not provide quantitative data that are predictive of sensitivity to therapeutic agents that target specific oncoprotein targets. Thus, an IHC test cannot determine whether or not a tumor cell population will be sensitive to treatment with an anti-EGFR agent.

Studies involving other IHC assays, such as the Her2 IHC test, suggest the results obtained from such tests may be wrong or misleading. This is likely because different laboratories use different rules for classifying positive and negative IHC status. Each pathologist running a test also may use different criteria to decide whether the results are positive or negative. In most cases, this happens when the test results are borderline, i.e. the results are neither strongly positive nor strongly negative. In other cases, tissue from one area of cancer tissue can test positive while tissue from a different area of the cancer tests negative.

Inaccurate IHC test results may mean that patients diagnosed with cancer do not receive the best possible care. If all or part of a cancer is positive for a specific target oncoprotein but test results classify it as negative, physicians are unlikely to implement the correct therapeutic treatment, even though the patient could potentially benefit from agents that target the oncoprotein. If a cancer is oncoprotein target negative but test results classify it as positive, physicians may use a specific therapeutic treatment, even though the patient is not only unlikely to receive any benefit but also is exposed to the agent's secondary risks.

Thus there is great clinical value in the ability to correctly evaluate quantitative levels of the KRAS protein in tumors, especially lung tumors, so that the patient will have the greatest chance of receiving a successful treatment regimen while reducing unnecessary toxicity and other side effects.

Determining quantitative levels of the KRAS fragment peptide is achieved using a mass spectrometer by the SRM/MRM methodology, in which the SRM/MRM signature chromatographic peak area of the peptide is determined within a complex peptide mixture present in a liquid tissue lysate (see U.S. Pat. No. 7,473,532, as described above). Quantitative levels of the analyzed protein is then determined by the SRM/MRM methodology whereby the SRM/MRM signature chromatographic peak area of an individual specified peptide from each of the proteins in one biological sample is compared to the SRM/MRM signature chromatographic peak area of a known amount of a “spiked” internal standard for each of the individual specified fragment peptides.

In one embodiment, the internal standard is a synthetic version of the same fragment peptides where the synthetic peptides contain one or more amino acid residues labeled with one or more heavy isotopes, such as ²H, ¹⁸O, ¹⁷O, ¹⁵N, ¹³C, or combinations thereof. Such isotope labeled internal standards are synthesized so that mass spectrometry analysis generates a predictable and consistent SRM/MRM signature chromatographic peak that is different and distinct from the native fragment peptide chromatographic signature peaks and which can be used as comparator peaks. Thus when the internal standard is “spiked” in known amounts into a protein or peptide preparation from a biological sample and analyzed by mass spectrometry, the SRM/MRM signature chromatographic peak area of the native peptide is compared to the SRM/MRM signature chromatographic peak area of the internal standard peptide, and this numerical comparison indicates either the absolute molarity and/or absolute weight of the native peptide present in the original protein preparation from the biological sample. Quantitative data for fragment peptides are displayed according to the amount of protein analyzed per sample.

In order to develop the SRM/MRM assay for the fragment peptide additional information beyond simply the peptide sequence needs to be utilized by the mass spectrometer. That additional information is used to direct and instruct the mass spectrometer, (e.g., a triple quadrupole mass spectrometer) to perform the correct and focused analysis of the specified fragment peptides. An SRM/MRM assay may be effectively performed on a triple quadrupole mass spectrometer. That type of a mass spectrometer may be considered to be one of the most suitable instruments for analyzing a single isolated target peptide within a very complex protein lysate that may consist of hundreds of thousands to millions of individual peptides from all the proteins contained within a cell. The additional information provides the mass spectrometer, such as a triple quadrupole mass spectrometer, with the correct directives to allow analysis of a single isolated target peptide within a very complex protein lysate. SRM/MRM assays also can be developed and performed on other types of mass spectrometer, including MALDI, ion trap, ion trap/quadrupole hybrid, or triple quadrupole instruments, but presently the most advantageous instrument platform for SRM/MRM assay is often considered to be a triple quadrupole instrument platform. The additional information about target peptides in general, and in particular about the specified fragment peptides, may include one or more of the mono isotopic mass of each peptide, its precursor charge state, the precursor m/z value, the m/z transition ions, and the ion type of each transition ion. The peptide sequence of the specified KRAS fragment peptide and the necessary additional information as described for this specified fragment peptide is shown in Table 1.

TABLE 1

| | | Mono | Precursor | Pre- | Trans- | |
|---|---|---|---|---|---|---|
| | Peptide | Isotopic | Charge | cursor | ition | Ion |
| SEQ ID | Sequence | Mass | State | ink | ink | type |
| SEQ ID NO: 1 | SFEDIHHYR | 1202.5468 | 2 | 602.281 | 475.241 | Y3 |
| | | | 2 | 602.281 | 612.3 | y4 |
| | | | 2 | 602.281 | 725.384 | Y5 |
| | | | 2 | 602.281 | 840.411 | y6 |
| | | | 2 | 602.281 | 969.453 | y7 |

To determine an appropriate reference level for protein quantitation, tumor samples were obtained from a cohort of patients suffering from cancer, in this case gastroesophageal adenocarcinoma. The tumor samples were formalin-fixed using standard methods and the level of KRAS in the samples was measured using the methods as described above. The tissue samples may also be examined using IHC and FISH using methods that are well known in the art. The patients in the cohort are treated with an anti-EGFR therapeutic agent and the response of the patients is measured using methods that are well known in the art, for example by recording the overall survival (OS) of the patients at time intervals after treatment. A suitable reference level is determined using statistical methods that are well known in the art, for example by determining the lowest p value of a log rank test. Once a reference level is determined it is used to identify those patients whose protein expression levels indicate that they may likely benefit from an anti-EGFR therapeutic regimen as measured by extending the life of the patient.

The skilled artisan will recognize that anti-EGFR agents may also be used as part of a treatment regimen that includes additional drugs or combinations of drugs. Levels of KRAS in patient tumor samples are typically expressed in amol/μg, although other units can be used. The skilled artisan will recognize that a reference level can be expressed as a range around a central value, for example, +/−250, 150, 100, 50 or 25 amol/μg. In the specific example described in detail below a suitable reference level for the KRAS protein was found to be 1331 amol/μg. However, the skilled artisan will recognize that levels higher or lower than these reference levels can be selected based on clinical results and experience.

Because both nucleic acids and protein can be analyzed from the same Liquid Tissue® biomolecular preparation it is possible to generate additional information about disease diagnosis and drug treatment decisions from the nucleic acids in same sample upon which proteins were analyzed. For example, if the KRAS protein is expressed by certain cells at increased levels, when assayed by SRM the data can provide information about the state of the cells and their potential for uncontrolled growth, choice of optimal therapy, and potential drug resistance. At the same time, information about the status of genes and/or the nucleic acids and proteins they encode (e.g., mRNA molecules and their expression levels or splice variants) can be obtained from nucleic acids present in the same Liquid Tissue® biomolecular preparation. Nucleic acids can be assessed simultaneously to the SRM analysis of proteins, including the KRAS protein. In one embodiment, information about KRAS protein and/or one, two, three, four or more additional proteins may be assessed by examining the nucleic acids encoding those proteins. Those nucleic acids can be examined, for example, by one or more, two or more, or three or more of: sequencing methods, polymerase chain reaction methods, restriction fragment polymorphism analysis, identification of deletions, insertions, and/or determinations of the presence of mutations, including but not limited to, single base pair polymorphisms, transitions, transversions, or combinations thereof.

### Example: Determination of a Predictive Value of Protein Expression Levels for Anti-EGFR Sensitivity in a Population of Gastroesophageal Adenocarcinoma Patients

### Methods

410 gastroesophageal cancer (GEC) samples and 30 cell lines were assessed for KRAS gene copy number (GCN) by fluorescence in situ hybridization (FISH) (n=90), KRAS expression by selected reaction monitoring mass spectrometry (KRAS-SRM-MS) (n=393), and KRAS-SRM level evaluated for correlation with KRAS amp+ status (n=73). Survival analysis was performed comparing KRAS amp+ versus non-amp+ patients. When possible, concurrent 315 gene next-generation sequencing was also performed. Four KRAS-amplified xenograft cell lines (CAT-2, 12, 14, 15, 17) were established from malignant effusions. Tumorigenic activity of KRAS amp+ lines (CAT lines, MKN-1) were assessed using MTT and soft agar assays in vitro and subcutaneous xenograft models, compared to non-amp+ lines. Inhibitory assays were performed using KRAS siRNA and CRIPSR, and commercial inhibitors targeting downstream effectors MEK and/or PIK3CA.

### Results

KRAS FISH revealed clustered gene amp+ in 28.9% (26/90); these patients had worse prognosis than non-amp+ patients. GCN significantly correlated with KRAS expression. All KRAS amp+ cell lines significantly overexpressed KRAS protein and were tumorigenic in xenograft subcutaneous models. KRAS siRNA and KRAS CRISPR of KRAS amp+ cell lines demonstrated inhibition in MTT viability and soft agar assays, compared to appropriate controls, and demonstrated significant and durable xenograft growth reduction. Conversely, inhibition using MEK and/or PI3K inhibitors demonstrated only transient growth reduction in vivo.

### CONCLUSIONS

KRAS gene amp+ was observed in a large subset (26%) of GEC patients, which correlated with extreme expression by mass spectrometry. Established xenograft lines serve as models to investigate therapeutic strategies for KRAS amp+ patients. Inhibition using MEK/PIK3CA inhibitors provided transient benefit for KRAS amp+ tumors while durable inhibition was observed with KRAS protein knockdown, suggesting benefit from siRNA therapeutics.

### Methods

Tumor cells from FFPE tumor tissue were procured and isolated from the tumor tissue by tissue microdissection and solubilized for downstream mass spectrometry analysis using the Liquid Tissue® reagents as described above. Protein levels were quantitated using selected reaction monitoring mass spectrometry (SRM-MS). Overall survival curves of the patients in this study as related to levels of various proteins, including KRAS, CAT, NQO1, XRCC1 and ECAD proteins, were developed.

1. A method of treating a patient suffering from cancer comprising:
(a) quantifying the level of a specified KRAS fragment peptide in a protein digest prepared from a tumor sample obtained from the patient and calculating the level of said KRAS peptide in said sample by selected reaction monitoring using mass spectrometry;
(b) comparing the level of said KRAS fragment peptide to a reference level, and
(c) treating the patient with a therapeutic regimen comprising an effective amount of at least one anti-EGFR agent when the level of the KRAS fragment peptide is lower than said reference level, and
(d) treating the patient with a therapeutic regimen that does not comprise an effective amount of at least one anti-EGFR agent when the level of the KRAS fragment peptide is above said reference level.

2. The method of claim 1 wherein said reference level of the KRAS fragment peptide is 1331 amol/μg., +/−250 amol/μg, of biological sample protein analyzed.

3. The method of claim 1 wherein said reference level is selected from the group consisting of 1331 amol/μg., +/−150 amol/μg, of biological sample protein analyzed, 1331 amol/μg., +/−100 amol/μg, of biological sample protein analyzed, 1331 amol/μg., +/−50 amol/μg, of biological sample protein analyzed and 1331 amol/μg., +/−25 amol/μg, of biological sample protein analyzed

4-6. (canceled)

7. The method of claim 1, wherein said protein digest of said biological sample is prepared by the Liquid Tissue protocol.

8. The method of claim 1, wherein said protein digest comprises a protease digest.

9. The method of claim 8, wherein said protein digest comprises a trypsin digest.

10. The method of claim 1, wherein mass spectrometry comprises tandem mass spectrometry, ion trap mass spectrometry, triple quadrupole mass spectrometry, MALDI-TOF mass spectrometry, MALDI mass spectrometry, hybrid ion trap/quadrupole mass spectrometry and/or time of flight mass spectrometry.

11. The method of claim 10, wherein the mode of mass spectrometry used is Selected Reaction Monitoring (SRM), Multiple Reaction Monitoring (MRM), Parallel Reaction Monitoring (PRM), intelligent Selected Reaction Monitoring (iSRM), and/or multiple Selected Reaction Monitoring (mSRM).

12. The method of claim 1, wherein the specified KRAS peptide has the amino acid sequence as set forth as SEQ ID NO:1.

13. The method of claim 1, wherein the tumor sample is a cell, collection of cells, or a solid tissue.

14. The method of claim 14, wherein the tumor sample is formalin fixed solid tissue.

15. The method of claim 15, wherein the tissue is paraffin embedded tissue.

16. The method of claim 1, wherein quantifying the specified KRAS fragment peptide comprises determining the amount of the KRAS peptide in said sample by comparing to a spiked internal standard peptide of known amount, wherein both the native peptide in the biological sample and the internal standard peptide corresponds to the same amino acid sequence of the KRAS fragment peptide as shown in SEQ ID NO:1.

17. The method of claim 1, wherein the internal standard peptide is an isotopically labeled peptide.

18. The method of claim 1, wherein the isotopically labeled internal standard peptide comprises one or more heavy stable isotopes selected from ¹⁸O, ¹⁷O, ¹⁵N, ¹³C, ²H or combinations thereof.

19. The method of claim 17, wherein detecting and quantitating the specified KRAS fragment peptide can be combined with detecting and quantitating other peptides from other proteins in a multiplex format so that the treatment decision about which agent used for treatment is based upon specific levels of the specified KRAS fragment peptide in combination with other peptides/proteins in the biological sample.

20. The method of claim 1 wherein: when said level of said specified KRAS peptide is lower than said reference level, then said therapeutic strategy comprises at least one agent selected from the group consisting of the cetuximab, panitumumab, zalutumumab, nimotuzumab, and matuzumab.

21. The method of claim 1, wherein said patient is suffering from gastroesophageal adenocarcinoma.
